(19) United States
(12) Patent Application Publication
(10) Pub. No.: US 2015/0090276 A1
(43) Pub. Date: Apr. 2, 2015

(54) SMOKING CESSATION DEVICE

(71) Applicant: Andrew Chunkil Park, Los Angeles, CA (US)

(72) Inventor: Andrew Chunkil Park, Los Angeles, CA (US)

(21) Appl. No.: 14/503,790

(22) Filed: Oct. 1, 2014

Related U.S. Application Data
(60) Provisional application No. 61/885,650, filed on Oct. 2, 2013.

(51) Int. Cl. A24F 47/00; A61K 9/68

(52) U.S. Cl. CPC A24F 47/002; A61K 9/0058
USPC 131/273

(57) ABSTRACT

A smoking cessation device for use in nicotine replacement therapy includes an imitation smoking implement with a body sized and shaped substantially identical to a conventional smoking implement chosen from the group consisting of a cigarette, a cigarillo, a cigar, and a pipe. The device also includes one or more nicotine replacement modules removably disposed at one end of the body, the one or more nicotine replacement modules removable from the imitation smoking implement by a user to ingest said nicotine replacement modules as part of a nicotine replacement therapy. Optionally, a plurality of said imitation smoking implements can be held in a package sized and shaped to resemble a conventional box of cigarettes, cigarillos or cigars. Said imitation smoking implement can be held by the user in their hands or lips while consuming the one or more nicotine replacement modules to simulate a conventional smoking ritual.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57.

The present application claims priority under 35 U.S.C. 119(e) to U.S. Provisional Patent Application No. 61/885,650 filed Oct. 2, 2013, the entire contents of which are hereby incorporated by reference and should be considered a part of this specification

### BACKGROUND

1. Field

The present invention is directed to a smoking cessation device for treating nicotine dependence from tobacco use, and, more particularly, to an imitation smoking implement with one or more removable nicotine replacement modules (e.g., nicotine gum pieces).

2. Description of the Related Art

According to the U.S. Food and Drug Administration (FDA), tobacco use is the leading preventable cause of death in the United States and is responsible for hundreds of thousands of deaths each year. Smoking tobacco can lead users to develop a variety of cancers, heart disease, stroke, pregnancy complications, chronic obstructive pulmonary disease, and other diseases that can shorten a smoker's life expectancy by 14 years or more.

Nicotine replacement therapy (NRT) pharmacologic agents are the most frequently used class of pharmacologic agents in tobacco dependence treatment. Several types of FDA-approved smoking cessation NRT agents are available, both with and without a prescription. NRT pharmacologic agents include gums, lozenges, sublingual tablets, skin patches, inhalers and nasal sprays. The FDA has approved the following NRT agents for purchase over-the-counter: nicotine gum, transdermal nicotine patches and nicotine lozenges.

Nicotine polacrilex gum is the most common type of nicotine gum used in NRT but other types of chewable nicotine gum exist. Nicotine gum has had some success as a smoking cessation aid, and studies including randomized control trials have shown that the odds of quitting tobacco usage is two to six times higher in active nicotine gum users versus placebo-treated users.

However, nicotine replacement therapy has not proven to be efficacious with users of all levels of nicotine dependence (e.g., with low-dependence users whom smoke fewer than about 10 cigarettes per day and/or as assessed by the Fagerstrom Test for Nicotine Dependence). This lack of proven efficacy could be at least partially explained by the failure of commercially available NRT agents to adequately recreate elements (e.g., psychologically gratifying elements) associated by smokers with the smoking ritual.

### SUMMARY

Accordingly, there is a need for an improved NRT device and method that recreates elements associated by smokers with the smoking ritual, where the device can deliver a dosage of nicotine to the user (e.g., nicotine gum of proven smoking cessation efficacy), to thereby improve the nicotine replacement therapy and smoking cessation rates.

In accordance with one aspect of the invention, an imitation smoking implement is provided that does not contain tobacco. The imitation smoking implement can be sized and shaped like a commercially available cigarette, cigarillo, or cigar and can have a cylindrical body defined by a filter made of filter tow, and can optionally be covered with rolling paper. One or more nicotine replacement modules can be removably affixed at an end of the body. In some variations, the nicotine replacement modules can be a nicotine gum piece.

Alternatively, the imitation smoking implement can be sized and shaped like a conventional pipe.

In accordance with another aspect of the invention, a package can be provided, the package sized and shaped to resemble a conventional box for cigarettes, cigarillos or cigars. The package can hold one or more imitation smoking implements, such as those as described above. Optionally, the package can include an accounting tool for aiding the user in keeping track of the number of imitation smoking implements he or she has used during an attempt to quit smoking.

In accordance with another aspect of the invention, a smoking cessation device for use in nicotine replacement therapy is provided. The device comprises an imitation smoking implement shaped and sized substantially identical to a conventional smoking implement chosen from the group consisting of a cigarette, a cigarillo, a cigar, and a pipe. The device also comprises one or more nicotine replacement modules removably disposed at one end of the imitation smoking implement, the one or more nicotine replacement modules removable from the imitation smoking implement by a user to ingest said nicotine replacement modules as part of a nicotine replacement therapy. Said imitation smoking implement can be held by the user in their hands or lips while consuming the one or more nicotine replacement modules to simulate a conventional smoking ritual.

In accordance with another aspect of the present invention, a smoking cessation kit for use in nicotine replacement therapy is provided. The kit comprises a package sized and shaped substantially identical to a conventional box for cigarettes, cigarillos or cigars. The kit also comprises one or more imitation smoking implements shaped and sized substantially identical to a conventional smoking implement chosen from the group consisting of a cigarette, a cigarillo, and a cigar. One or more nicotine replacement modules is removably disposed at one end of at least one of the one or more imitation smoking implements, the one or more nicotine replacement modules removable from said imitation smoking implement by a user to ingest said nicotine replacement modules as part of a nicotine replacement therapy. One or both of said package and imitation smoking implements can be held by the user while consuming the one or more nicotine replacement modules so as to simulate a conventional smoking ritual.

In accordance with another aspect of the present invention, a nicotine replacement module is provided that is composed of a mixture of nicotine, a porous carrier (e.g., a carrier that has a solid form, and preferably a particulate form) and at least one other non-active ingredient.

These and other objects, features and advantages of the present invention will become more apparent from the detailed description of the embodiment(s) below when read in conjunction with the drawings.

### BRIEF DESCRIPTION OF THE DRAWINGS

FIG. 1.1 is a schematic exploded perspective view of one embodiment of an imitation smoking implement with one embodiment of a removable nicotine replacement module.

FIG. 1.2 is a schematic front view of the imitation smoking implement of FIG. 1.1.

FIG. 1.3 is a schematic side view of the imitation smoking implement of FIG. 1.1, a sectional portion thereof showing the positioning of the nicotine replacement module relative to the imitation smoking implement.

FIG. 2.1 is a schematic front view of one embodiment of a package for holding multiple imitation smoking implements, such as the imitation smoking implement in FIG. 1.1.

FIG. 2.2 is a schematic side view of the package of FIG. 2.1 with the top of the package closed.

FIG. 2.3 is a schematic side view of the package of FIG. 2.1 with the top of the package open to allow access to one or more imitation smoking implements.

FIG. 2.4 is a bottom view of the package of FIG. 2.1.

FIG. 3 shows a schematic side view of another embodiment of an imitation smoking implement with a removable nicotine replacement module.

FIG. 4 shows a schematic side view of another embodiment of an imitation smoking implement with a removable nicotine replacement module.

FIG. 5.1 shows a schematic side view of another embodiment of an imitation smoking implement with a removable nicotine replacement module, a sectional portion thereof showing the positioning of the removable nicotine replacement module relative to the imitation smoking implement.

FIG. 5.2 shows a schematic side view of the imitation smoking implement of FIG. 5.1 with another embodiment of a removable nicotine replacement module.

FIG. 5.3 shows a schematic side view of the imitation smoking implement of FIG. 5.1 with another embodiment of a removable nicotine replacement module.

FIG. 6.1 shows a schematic side view of another embodiment of an imitation smoking implement with a removable nicotine replacement module.

FIG. 6.2 shows a schematic side view of the imitation smoking implement FIG. 6.1 with another embodiment of a removable nicotine replacement module.

FIG. 6.3 shows a schematic side view of the imitation smoking implement FIG. 6.1 with another embodiment of a removable nicotine replacement module.

FIGS. 7A-7C shows a schematic perspective view of another embodiment of an imitation smoking implement housing a plurality of removable nicotine replacement modules.

FIG. 8 shows a method of using one or more imitation smoking implements with removable nicotine replacement modules.

### DETAILED DESCRIPTION

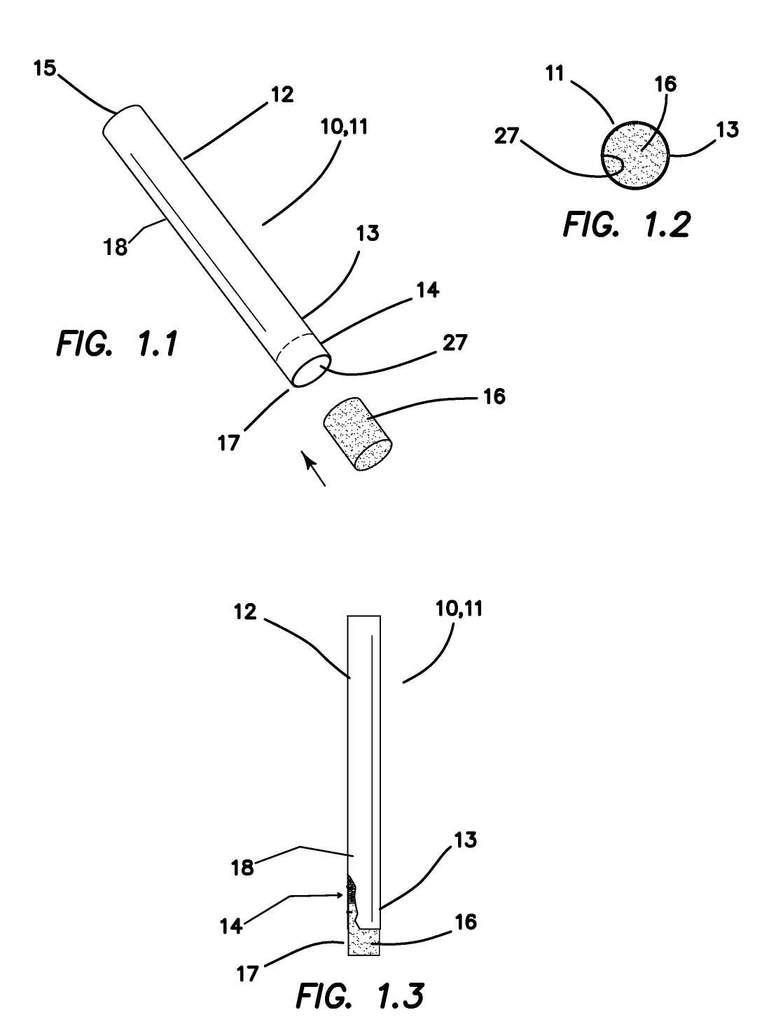

FIGS. 1.1-1.3 show one embodiment of an imitation smoking implement 10 for use in nicotine replacement therapy (NRT) for smoking cessation. In the illustrated embodiment, the imitation smoking implement 10 can be an imitation cigarette 11 or Q-cigarette (e.g., cylindrically shaped like a commercially available cigarette). The imitation smoking implement 10 has a cylindrical body 12 of approximately the same dimensions as a conventional cigarette. For example, in one embodiment, the cylindrical body 12 can have a length of about three inches and an outer diameter of about 5/16 inches. In other embodiments, the imitation smoking implement 10 has approximately the same dimensions as a conventional cigarillo or cigar.

The imitation smoking implement 10 has a filter 13. The filter 13 can be made of filter tow 14 that defines the body of the filter 13. The filter tow 14 can optionally be made of a cellulose acetate material and/or be biodegradable. In the illustrated embodiment, the filter 13 extends from the proximal end 15 toward the distal end 17 of the imitation smoking implement 10 (see dashed line in FIG. 1.1). The filter 13 can have a length of about 2¾ inches and a diameter of about 5/16 inches.

The body 12 can be covered by rolling paper 18, such as that used in conventional cigarettes, such that the imitation smoking implement 10 has as similar look, feel and texture as a cigarette. The rolling paper 18 can have dimensions of about 3¼ inches by about 1 1/10 inches and wrapped around the body 12. However, the rolling paper 18 can have other suitable dimensions. The rolling paper 18 can be lined with an adhesive (e.g., cornstarch adhesive) to seal the filter tow 14. The paper 18 can cover the body 12 and extend between the proximal end 15 and past an end of the filter 13 so as to define a cavity or pocket 27 in the distal end of the body 12. In one embodiment, the rolling paper 18 can extend approximately ¼inch past the end of the filter 13. In another embodiment, the rolling paper can extend approximately ½ inch past the end of the filter 13. The filter 13 can also have a recess near the distal end 17 of the body 12 that defines at least a portion of the cavity or pocket 27. The cavity or pocket 27 can be cylindrical in shape and sized to removably receive one or more nicotine replacement modules 16 therein. In the embodiment illustrated in FIGS. 1.1-1.3, one nicotine replacement module 16 is removably disposed in the cavity or pocket 27, which can optionally extend at least partially past the end of the paper 18 (see FIG. 1.3) to be accessible to a user's teeth or fingers for consumption. Optionally, the nicotine replacement module 16 can be generally cylindrical (e.g., have a cylindrical outer surface), have a length of about ½ inch and a diameter of about 5/16 inches (e.g., have substantially the same diameter as the rest of the imitation smoking implement 10), such that about half of the nicotine replacement module 16 is disposed in the cavity or pocket 27. However, as described further below, the cavity or pocket 27 can be sized to removably hold a plurality of (e.g., two or more) nicotine replacement modules 16 therein.

With continued reference to FIGS. 1.1-1.3, the nicotine replacement module 16 can be a piece of nicotine gum that can be cylindrically shaped along at least a portion of its length to at least partially fit within the cavity or pocket 27. As shown in FIG. 1.3, a portion of the nicotine replacement module 16 can extend from the distal end of the cavity or pocket 27 defined by the rolling paper 18. Each nicotine replacement module 16 can deliver a dosage (e.g., 2 mg, 4 mg) of nicotine as part of a nicotine replacement therapy, as discussed herein. Where the nicotine replacement module 16 is nicotine gum, it can be nicotine polacrilex gum (e.g., Nicorette®, Thrive®, Nicotinell®) or other suitable type of nicotine gum (e.g., Zonnic®). Though the nicotine replacement module 16 is described as being nicotine gum, in other embodiments the nicotine replacement module 16 can instead be a lozenge, pill, liquid or gel cap, or other type of module for delivering nicotine in solid or liquid form.

During use, the user could hold the proximal end 15 of the body 12 in their mouth until ready to ingest the nicotine replacement module 16, at which point the user could take the body 12 out of their mouth, remove the nicotine replacement module 16 from the distal end 17 of the body 12 (e.g., with their teeth, fingers) and place it in their mouth. Where the nicotine replacement module 16 is nicotine gum, the user can alternate between chewing the nicotine gum and holding the gum between their cheek and gum in order to extract the maximum amount of nicotine from the gum. Advantageously, the user could continue to use the imitation smoking implement 10 like a cigarette, such as continue to hold it in their hand or continue to place it in their mouth, all the while receiving nicotine from the nicotine replacement module 16. Accordingly, the imitation smoking implement 10 would satisfy the user's need and/or desire to experience other aspects of smoking (e.g., holding a cigarette-like device with their hands, lips).

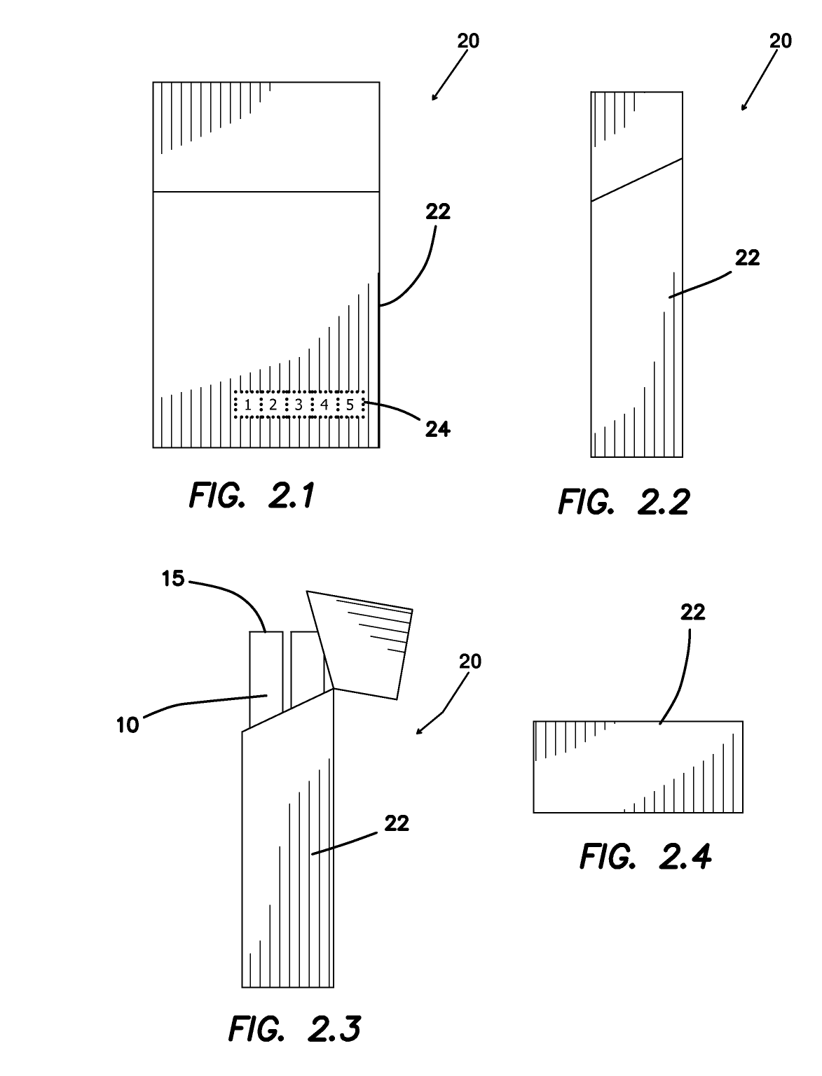

FIGS. 2.1-2.4 show one embodiment of a package 20 for removably housing one or more imitation smoking implements 10. The package 20 can be a front-opening flip-top cardboard box 22, similar in size and shape to packaging for commercially available cigarettes, such as Marlboro® brand cigarettes. In one embodiment, the box 22 can be about 3½ inches tall, about 2⅙ inches wide and about ⅞ inches deep. However, the box 22 can have other suitable dimensions. The package 20 advantageously allows the user to interact with the imitation smoking implements 10 in a similar manner as conventional cigarettes (e.g., by carrying a similarly sized and shaped package, for example in their shirt pocket, and unpacking and removing the implements 10 from the package 20 in a similar manner as cigarettes) to thereby imitate the smoking experience. For example, as shown in FIG. 2.3, the user can open the top of the box 22 and remove an imitation smoking implement 10 from the box 22 by its proximal end 15, just as they would with a conventional cigarette.

The box 22 can optionally include an accounting tool 24 to aid the user in tracking the number of imitation smoking implements 10 they have used in the course of an attempt to quit smoking. As shown in FIG. 2.1, the accounting tool 24 can be a plurality of numbered squares or tabs that the user can mark or puncture as they take an imitation smoking implement 10 out of the box 22, or after using said implement 10. Optionally, the squares or tabs can have perforated edges to facilitate the user tearing away or puncturing the particular square or tab.

Optionally, the box 22 includes instructions (e.g., on a surface of the box, in a slip of paper inserted in the box 22) for the user on how to use the imitation smoking implements 10. Advantageously, such instructions instruct the smoker to interact with the imitation smoking implements 10 in the same manner as a cigarette (e.g., holding it in their fingers, placing the proximal end 15 in their mouth).

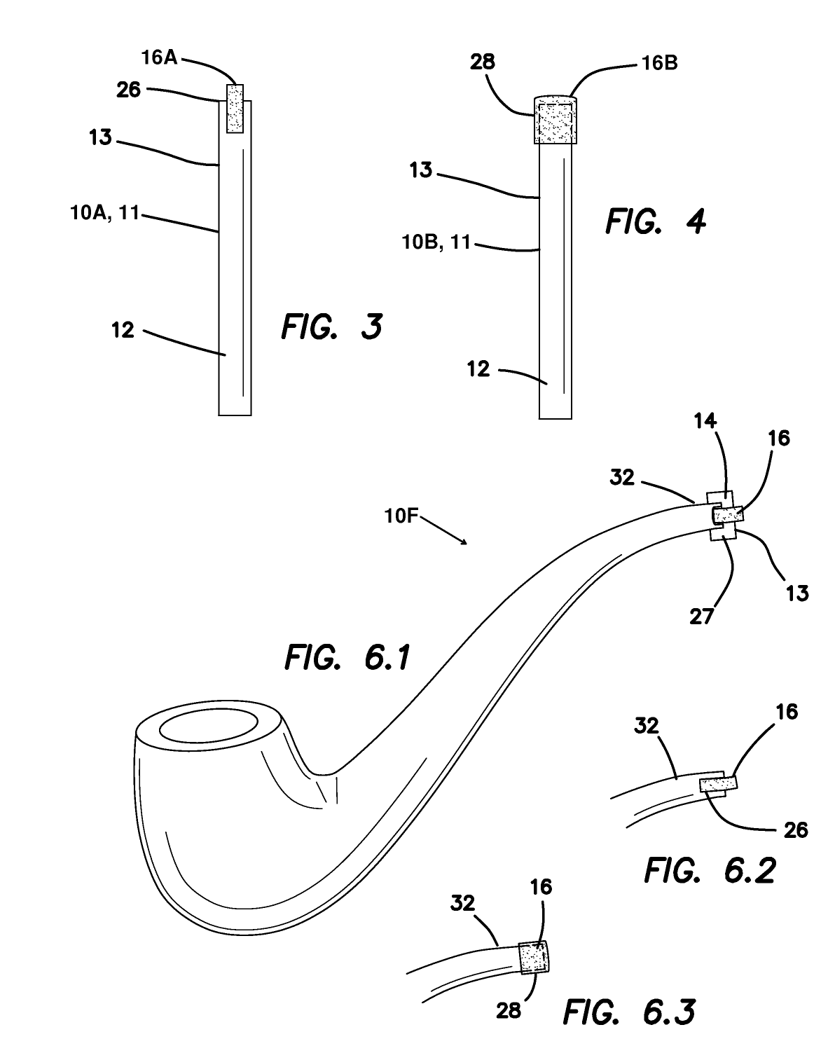

FIG. 3 shows another embodiment of an imitation smoking implement 10A. The imitation smoking implement 10A is constructed similar to the imitation smoking implement 10 shown in FIGS. 1.1-1.3, except as noted below. Thus, the reference numerals used to designate the various components of the imitation smoking implement 10A are identical to those used for identifying the corresponding components of the imitation smoking implement 10 in FIGS. 1.1-1.3, except that an “A” has been added to the reference numerals.

In the illustrated embodiment, the nicotine replacement module 16A is held solely by a recess 26 in the filter 13. Optionally, at least a portion of the nicotine replacement module 16A can be press-fit into the recess 26. In this embodiment, the rolling paper does not extend past the end of the filter 13 as with the embodiment in FIG. 1.1, or the rolling paper can be excluded altogether. The user can remove the nicotine replacement module 16A with their teeth, lips or fingers, and use the body 12 of the imitation smoking implement 10 as discussed above to imitate the smoking experience.

FIG. 4 shows another embodiment of an imitation smoking implement 10B. The imitation smoking implement 10B is constructed similar to the imitation smoking implement 10 shown in FIGS. 1.1-1.3, except as noted below. Thus, the reference numerals used to designate the various components of the imitation smoking implement 10B are identical to those used for identifying the corresponding components of the imitation smoking implement 10 in FIGS. 1.1-1.3, except that a “B” has been added to the reference numerals.

In the illustrated embodiment, the nicotine replacement module 16B is shaped to fit over the distal end of the body 12. Optionally, the nicotine replacement module 16B can be shaped like a cap 28. The nicotine replacement module 16B can be press-fit over the distal end of the body 12. Alternatively, the nicotine replacement module 16B can be adhered (e.g., with a cornstarch adhesive) to the distal end of the body 12. In the illustrated embodiment, the rolling paper 18 can optionally be excluded so that the nicotine replacement module fits over the filter 13 of the imitation smoking implement 10B. Optionally, the imitation smoking implement 10B can include the nicotine replacement module 16B (as the cap 28) and the nicotine replacement module 16 (see FIG. 1.1) or nicotine replacement module 16A (see FIG. 3), so that two nicotine replacement modules are provided in the imitation smoking implement, where the second nicotine replacement module is exposed or can otherwise be accessed once the cap 28 has been removed.

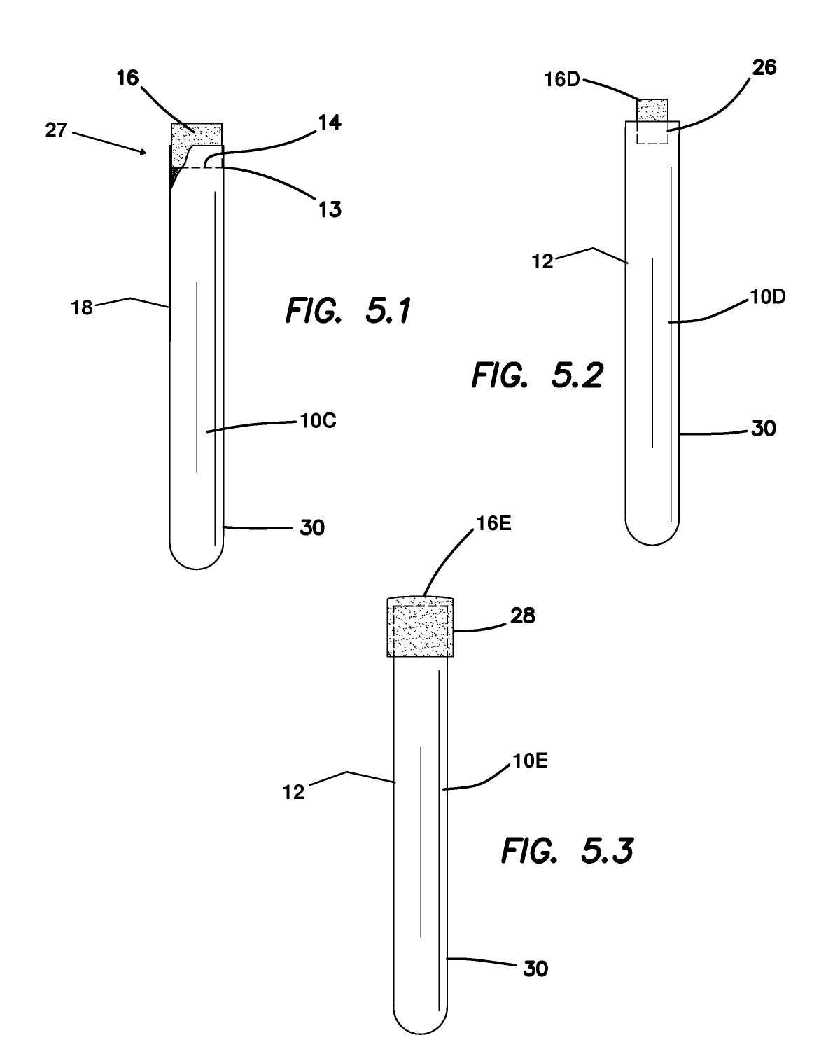

FIG. 5.1 shows another embodiment of an imitation smoking implement 10C. The imitation smoking implement 10C is constructed similar to the imitation smoking implement 10 shown in FIGS. 1.1-1.3, except as noted below. Thus, the reference numerals used to designate the various components of the imitation smoking implement 10C are identical to those used for identifying the corresponding components of the imitation smoking implement 10 in FIGS. 1.1-1.3, except that a “C” has been added to the reference numerals.

In the illustrated embodiment, the imitation smoking implement 10C is shaped like a cigar 30 and has a nicotine replacement module 16 that is removably housed in a cavity or pocket 27 at least partially defined by rolling paper 18 in a distal end of the cigar 30, in the same manner as described above in connection with the imitation smoking implement 10 of FIGS. 1.1-1.3.

FIG. 5.2 shows another embodiment of an imitation smoking implement 10D. The imitation smoking implement 10D is constructed similar to the imitation smoking implement 10A shown in FIG. 3, except as noted below. Thus, the reference numerals used to designate the various components of the imitation smoking implement 10D are identical to those used for identifying the corresponding components of the imitation smoking implement 10A in FIG. 3, except that a “D” has been added to the reference numerals instead of the “A”.

In the illustrated embodiment, the imitation smoking implement 10D is shaped like a cigar 30 and has a nicotine replacement module 16D is held solely by a recess 26 in the body 12 of the cigar 30. Optionally, at least a portion of the nicotine replacement module 16D can be press-fit into the recess 26. In this embodiment, the rolling paper can be excluded altogether. The user can remove the nicotine replacement module 16D with their teeth, lips or fingers, and use the cigar 30 body of the imitation smoking implement 10D as discussed above to imitate the smoking experience.

FIG. 5.3 shows another embodiment of an imitation smoking implement 10E. The imitation smoking implement 10E is constructed similar to the imitation smoking implement 10B shown in FIG. 4, except as noted below. Thus, the reference numerals used to designate the various components of the imitation smoking implement 10E are identical to those used for identifying the corresponding components of the imitation smoking implement 10B in FIG. 4, except that an “E” has been added to the reference numerals instead of the “B”.

In the illustrated embodiment, the imitation smoking implement 10E is shaped like a cigar 30 and has a nicotine replacement module 16E shaped to fit over the distal end of the body 12. Optionally, the nicotine replacement module 16E can be shaped like a cap 28. The nicotine replacement module 16E can be press-fit over the distal end of the body 12. Alternatively, the nicotine replacement module 16E can be adhered (e.g., with a cornstarch adhesive) to the distal end of the body 12. In the illustrated embodiment, the rolling paper, such as rolling paper 18, can optionally be excluded so that the nicotine replacement module fits over the filter 13 of the imitation smoking implement 10E. Optionally, the imitation smoking implement 10E can include the nicotine replacement module 16E (as the cap 28) and the nicotine replacement module 16 (see FIG. 5.1) or nicotine replacement module 16D (see FIG. 5.2), so that two nicotine replacement modules are provided in the imitation smoking implement, where the second nicotine replacement module is exposed or can otherwise be accessed once the cap 28 has been removed.

FIGS. 6.1-6.3 show another embodiment of an imitation smoking implement 10F. In the illustrated embodiment, the imitation smoking implement 10F is an imitation pipe 32. FIG. 6.1 shows one embodiment, where the nicotine replacement module 16 is affixed at least partially in a recess within the proximal end of the pipe 32. Rolling paper 14 can partially cover the proximal end of the pipe 32 and hold the nicotine replacement module 16 in place. FIG. 6.2 shows another embodiment of the pipe 32, where the nicotine replacement module 16 is held solely by a recess 26 in the proximal end of the pipe 32. Optionally, the nicotine replacement module 16 can be press-fit into the recess 26. In this embodiment, the rolling paper can be excluded. FIG. 6.3 shows another embodiment of the pipe 32. In this embodiment, the nicotine replacement module 16 can be affixed over the proximal end of the pipe 32. Optionally, the nicotine replacement module 16 can be shaped like a cap 28 and can be press-fit or adhered to the proximal end of the pipe 32. In one embodiment, the cap 28 can at least partially cover another nicotine replacement module, so that two nicotine replacement modules are provided in the pipe 32, where the second nicotine replacement module is exposed or can otherwise be accessed once the cap 28 has been removed.

During use, the user could remove the nicotine replacement module 16 (e.g., with their teeth, lips or fingers), place it in their mouth, and continue to hold the pipe with their hand or mouth to imitate the pipe smoking experience.

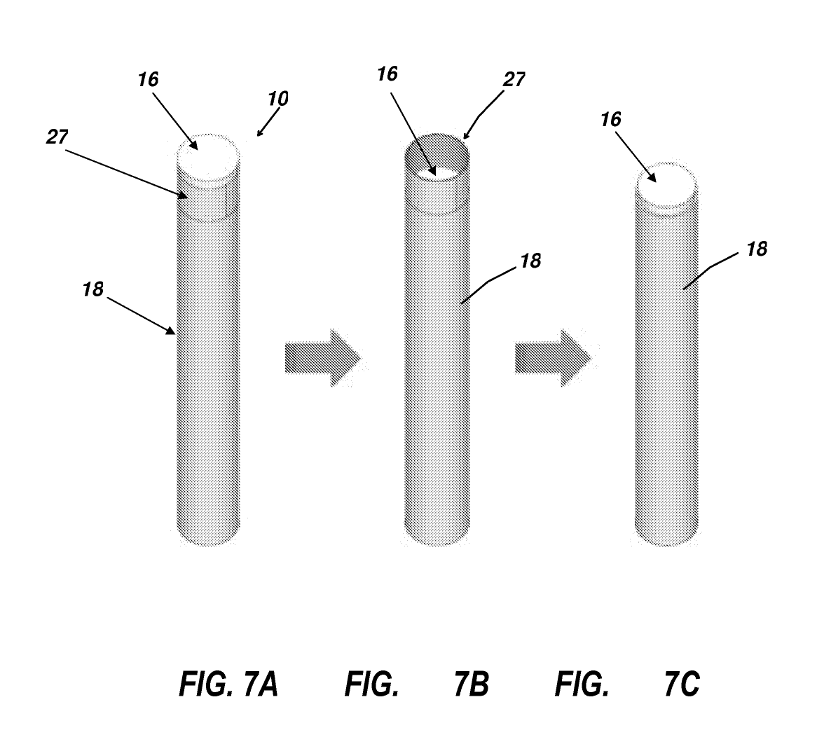

FIGS. 7A-7C show another embodiment of an imitation smoking implement 10F. The imitation smoking implement 10F is constructed similar to the imitation smoking implement 10 shown in FIGS. 1.1-1.3, except as noted below. Thus, the reference numerals used to designate the various components of the imitation smoking implement 10F are identical to those used for identifying the corresponding components of the imitation smoking implement 10 in FIGS. 1.1-1.3, except that an “F” has been added to the reference numerals.

In the illustrated embodiment, the imitation smoking implement 10F removably houses multiple (e.g., two or more) nicotine replacement modules 16. The imitation smoking implement 10F in FIG. 7A is similar to the imitation smoking implement in FIGS. 1.1-1.3 and the nicotine replacement module 16 is held in the same way (e.g., within a cavity or pocket 27 by the rolling paper 18). The user can consume one nicotine replacement module 16, and can thereafter access an additional nicotine replacement module 16 (see FIG. 7B) by removing a portion of the rolling paper 18. Optionally, the rolling paper can be perforated or scored to facilitate removal of a portion of the paper 18 to expose said additional nicotine replacement module 16 (see FIG. 7C).

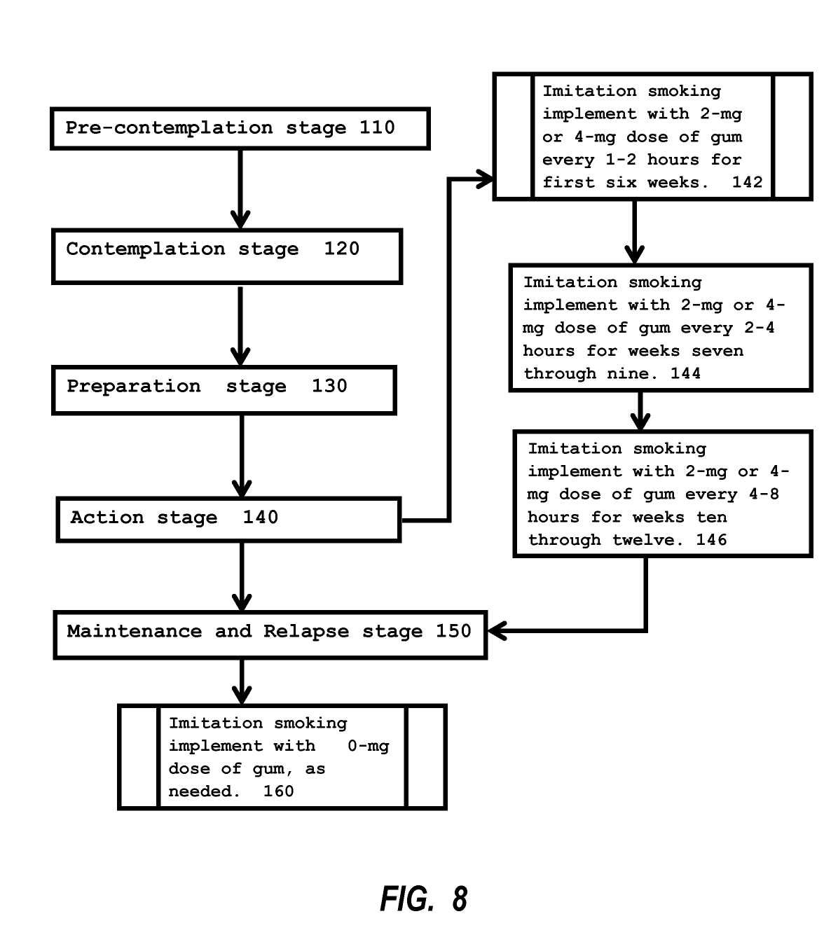

FIG. 8 shows a method 100 of using the imitation smoking implements 10 described herein in nicotine replacement therapy. Five discrete stages in a typical smoking cessation process include: the Pre-contemplation stage 110, or the act of becoming aware of the benefits of quitting smoking; and the Contemplation stage 120, or the act of beginning to think about quitting though not being quite ready to quit because a person is unwilling to give up the pleasure or comfort of tobacco. Transition from pre-contemplation to contemplation often involves recognition by a person that he or she is experiencing harm from tobacco use and there is social pressure exerted by family, peers and employers to quit. Public knowledge of efficacious tobacco dependence treatment may influence a potential user's social group to begin exerting this pressure. During the Contemplation stage, a person weighs the benefits of continued tobacco use against its costs. The present invention advantageously recreates the smoking ritual from point-of-sale to nicotine consumption. Its features are designed to decrease the perceived cost of quitting tobacco use. In the Preparation stage 130, a person makes a firm decision to take the necessary steps to quit and makes a self-assessment about his or her nicotine consumption habits. Selection of quitting method and a planned response to anticipated situations, unanticipated situations and stressors are components of the Preparation stage. The embodiments disclosed herein replace smoking behaviors and user serum nicotine levels and therefore are a viable preparatory tool. In the Action stage 140, where a person is actively not smoking despite any urge to do so, the imitation smoking implements 10 are used to taper nicotine consumption according to established guidelines for nicotine gum. The tapering regimen is an evidence-based therapy for the Action stage. In the Maintenance and Relapse stage 150, a person continues to avoid the temptation to smoke again and reinitiates the addictive habit, respectively. In some instances, a person may abstain from smoking indefinitely and not experience a relapse. Even when NRT users in the Action stage 140 of smoking cessation are not experiencing frank tobacco cravings, elements of the smoking ritual and associated behaviors such as drinking coffee, driving and socializing may trigger relapse to habitual, active smoking. In a now-former smoker's final stage of maintenance, zero-dose (0-mg nicotine) imitation smoking implements 10 containing gum 16 absent any nicotine may be used indefinitely 160 to prevent relapse.

This commonly accepted protocol, a method of nicotine replacement therapy, recommends a user choose a day to quit smoking, and then to consume a piece of nicotine gum when a user feels the urge to smoke during the Action stage 140. Consumption by the park-and-chew method is recommended whereby a user chews the gum until a tingling is felt, then parks it in their buccal mucosa until the tingling passes, and then repeats the process for a total duration of about 30 minutes. The dosage depends on the existing smoking habits of a user. For a person who habitually smokes within 30 minutes of waking, a 4-mg nicotine dose of gum is recommended. Otherwise a 2-mg dose is recommended. The regimen for use of nicotine gum is about 12 weeks. What has been found to work best for most users is one piece of gum consumed every one to two hours for the first six weeks of use 142, then one piece every two to four hours for weeks seven through nine 144, and then tapering to one piece every four to eight hours for weeks ten through twelve 146. As tapering of dosage progresses, a user becomes less physically dependent on nicotine. A user then is mostly or exclusively psychologically dependent on nicotine though they may continue to experience cravings.

The embodiments described herein advantageously provide an imitation smoking implement 10 (and optionally a package 20) that resemble commercially available smoking devices, allowing the user to replicate or simulate the smoking ritual by allowing the user to hold the imitation smoking implement 10 (e.g., with their fingers, in their mouths) and/or package 20 while consuming the nicotine replacement modules 16. As part of a nicotine replacement therapy (NRT), the embodiments disclosed herein provide a behaviorally and psychologically active NRT system that can aid the user in their attempt to quit smoking by allowing the user to perform as much of the smoking ritual as they are accustomed to while not actually smoking tobacco.

While certain embodiments of the inventions have been described, these embodiments have been presented by way of example only, and are not intended to limit the scope of the disclosure. Indeed, the novel methods and systems described herein may be embodied in a variety of other forms. For example, the construction of the imitation smoking implement 10 does not have to imitate a real smoking implement (e.g., cigarette, cigar, cigarillo) and other designs may be used. Additionally the components (e.g., nicotine replacement modules 16) of the imitation smoking implement 10 can be sold as a kit to be used with existing smoking implements (e.g., the filter 13 sold separately to be matched with a nicotine replacement therapy drug or module 16, such as gum, supplied by the user). Furthermore, various omissions, substitutions and changes in the systems and methods described herein may be made without departing from the spirit of the disclosure. The accompanying claims and their equivalents are intended to cover such forms or modifications as would fall within the scope and spirit of the disclosure. Accordingly, the scope of the present invention is defined only by reference to the appended claims.

Features, materials, characteristics, or groups described in conjunction with a particular aspect, embodiment, or example are to be understood to be applicable to any other aspect, embodiment or example described in this section or elsewhere in this specification unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The protection is not restricted to the details of any foregoing embodiments. The protection extends to any novel one, or any novel combination, of the features disclosed in this specification (including any accompanying claims, abstract and drawings), or to any novel one, or any novel combination, of the steps of any method or process so disclosed.

Furthermore, certain features that are described in this disclosure in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations, one or more features from a claimed combination can, in some cases, be excised from the combination, and the combination may be claimed as a subcombination or variation of a sub combination.

Moreover, while operations may be depicted in the drawings or described in the specification in a particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Further, the operations may be rearranged or reordered in other implementations. Those skilled in the art will appreciate that in some embodiments, the actual steps taken in the processes illustrated and/or disclosed may differ from those shown in the figures. Depending on the embodiment, certain of the steps described above may be removed, others may be added. Furthermore, the features and attributes of the specific embodiments disclosed above may be combined in different ways to form additional embodiments, all of which fall within the scope of the present disclosure. Also, the separation of various system components in the implementations described above should not be understood as requiring such separation in all implementations, and it should be understood that the described components and systems can generally be integrated together in a single product or packaged into multiple products.

For purposes of this disclosure, certain aspects, advantages, and novel features are described herein. Not necessarily all such advantages may be achieved in accordance with any particular embodiment. Thus, for example, those skilled in the art will recognize that the disclosure may be embodied or carried out in a manner that achieves one advantage or a group of advantages as taught herein without necessarily achieving other advantages as may be taught or suggested herein.

Conditional language, such as “can,” “could,” “might,” or “may,” unless specifically stated otherwise, or otherwise understood within the context as used, is generally intended to convey that certain embodiments include, while other embodiments do not include, certain features, elements, and/or steps. Thus, such conditional language is not generally intended to imply that features, elements, and/or steps are in any way required for one or more embodiments or that one or more embodiments necessarily include logic for deciding, with or without user input or prompting, whether these features, elements, and/or steps are included or are to be performed in any particular embodiment.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount. As another example, in certain embodiments, the terms “generally parallel” and “substantially parallel” refer to a value, amount, or characteristic that departs from exactly parallel by less than or equal to 15 degrees, 10 degrees, 5 degrees, 3 degrees, 1 degree, or 0.1 degree.

The scope of the present disclosure is not intended to be limited by the specific disclosures of preferred embodiments in this section or elsewhere in this specification, and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

What is claimed is:

1. A smoking cessation device for use in nicotine replacement therapy, comprising:
an imitation smoking implement shaped and sized substantially identical to a conventional smoking implement chosen from the group consisting of a cigarette, a cigarillo, a cigar, and a pipe; and
one or more nicotine replacement modules removably disposed at one end of the imitation smoking implement, the one or more nicotine replacement modules removable from the imitation smoking implement by a user to ingest said nicotine replacement modules as part of a nicotine replacement therapy,
wherein said imitation smoking implement can be held by the user in their hands or lips while consuming the one or more nicotine replacement modules so as to simulate a conventional smoking ritual.

2. The smoking cessation device of claim 1, wherein the one or more nicotine replacement modules comprises one or more nicotine gum pieces.

3. The smoking cessation device of claim 2, wherein the one or more nicotine gum pieces comprise two nicotine gum pieces disposed in line at said end of the imitation smoking implement, wherein the user can selectively access each of the two nicotine gum pieces during use of the imitation smoking implement.

4. The smoking cessation device of claim 3, wherein the user can selectively access each of the two nicotine gum pieces by peeling off at least a portion of a rolling paper that covers a body of the imitation smoking implement.

5. The smoking cessation device of claim 2, wherein at least a portion of the one or more nicotine gum pieces has a cylindrical outer surface.

6. The smoking cessation device of claim 5, wherein the one or more nicotine gum pieces are at least partially covered by a rolling paper that covers a body of the imitation smoking implement, said one or more nicotine gum pieces accessible by peeling off at least a portion of the rolling paper.

7. The smoking cessation device of claim 6, wherein said at least a portion of the rolling paper has a perforated or scored boundary.

8. The smoking cessation device of claim 5, wherein the one or more nicotine gum pieces are at least partially disposed in a recess within the body of the imitation smoking implement.

9. The smoking cessation device of claim 8, wherein at least a portion of the one or more nicotine gum pieces are press-fit into said recess.

10. The smoking cessation device of claim 5, wherein said body comprises a filter made of filter tow.

11. The smoking cessation device of claim 5, wherein at least one of the one or more nicotine gum pieces defines a cap configured to releasably fit over a body of the imitation smoking implement.

12. A smoking cessation kit for use in nicotine replacement therapy, comprising:
a package sized and shaped substantially identical to a conventional box for cigarettes, cigarillos or cigars; and
one or more imitation smoking implements shaped and sized substantially identical to a conventional smoking implement chosen from the group consisting of a cigarette, a cigarillo, and a cigar; and
one or more nicotine replacement modules removably disposed at one end of at least one of the one or more imitation smoking implements, the one or more nicotine replacement modules removable from said imitation smoking implement by a user to ingest said nicotine replacement modules as part of a nicotine replacement therapy,
wherein one or both of said package and imitation smoking implements can be held by the user while consuming the one or more nicotine replacement modules so as to simulate a conventional smoking ritual.

13. The smoking cessation device of claim 12, wherein the one or more nicotine replacement modules comprises one or more nicotine gum pieces.

14. The smoking cessation kit of claim 13, wherein at least a portion of the one or more nicotine gum pieces has a cylindrical outer surface.

15. The smoking cessation kit of claim 14, wherein the one or more nicotine gum pieces are at least partially covered by a rolling paper that covers a body of the imitation smoking implement, said one or more nicotine gum pieces accessible by peeling off at least a portion of the rolling paper.

16. The smoking cessation kit of claim 15, wherein said at least a portion of the rolling paper has a perforated or scored boundary.

17. The smoking cessation kit of claim 14, wherein the one or more nicotine gum pieces are at least partially disposed in a recess within a body of the imitation smoking implement.

18. The smoking cessation kit of claim 17, wherein said body comprises a filter made of filter tow.

19. The smoking cessation kit of claim 14, wherein at least one of the one or more nicotine gum pieces defines a cap configured to releasably fit over a body of the imitation smoking implement.

20. The smoking cessation kit of claim 12, wherein the package is a flip-top box and comprises an accounting tool on a surface of the box configured to aid the user in keeping track of the imitation smoking implements used.
